(19) United States
(12) Patent Application Publication
(10) Pub. No.: US 2021/0353745 A1
(43) Pub. Date: Nov. 18, 2021

(54) METHODS AND REAGENTS TO TREAT ALLERGY

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 17/385,908

(22) Filed: Jul. 27, 2021

Related U.S. Application Data
(63) Continuation of application No. 16/819,168, filed on Mar. 16, 2020, now abandoned.
(60) Provisional application No. 62/827,159, filed on Mar. 31, 2019.

(51) Int. Cl. A61K 39/35; A61K 39/39; A61K 31/4745; A61K 39/36; A61K 31/713; A61K 9/70

(52) U.S. Cl. CPC A61K 39/35; A61K 39/39; A61K 9/7023; A61K 39/36; A61K 31/713; A61K 31/4745

(57) ABSTRACT

Compositions, reagents and methods to treat allergy are described. In one aspect, the compositions and reagents involve an oral mucosa delivery system comprising an antigen causing said condition and an immune activity enhancing agent. The methods and regents to treat allergy also relate to applying the combination of allergen and immune activity enhancing agent either as a physical mixture or as synthetic conjugate to a subject in need.

### CROSS-REFERENCE TO RELATED APPLICATIONS

This application is a continuation application of U.S. patent application Ser. No. 16/819,168, filed on Mar. 16, 2020, which claims benefit of and priority to U.S. Provisional Patent Application 62/827,159 filed on Mar. 31, 2019. The entire disclosure of the prior application is considered to be part of the disclosure of the instant application and is hereby incorporated by reference.

### BACKGROUND OF THE INVENTION

### Field of the Invention

The current invention relates to reagents to treat auto immune disease and allergy. The current invention discloses methods to treat auto immune disease and allergy. In one aspect, the compositions and reagents involve a transdermal drug delivery system such as a transdermal patch to treat allergy comprising an antigen causing said condition and an immune activity enhancing agent. The methods and regents to treat allergy also relate to applying the combination of allergen and immune activity enhancing agent either as a physical mixture or as synthetic conjugate to a subject in need.

### Background Information

Immune responses are necessary for protection against potentially pathogenic microorganisms. However, undesired immune activation can cause injurious processes leading to damage or destruction of one's own tissues. Undesired immune activation occurs, for example, in autoimmune diseases where antibodies and/or T lymphocytes react with self antigens to the detriment of the body's tissues. This is also the case in allergic reactions characterized by an exaggerated immune response to certain environmental matters and which may result in inflammatory responses leading to tissue destruction. This is also the case in rejection of transplanted organs which is significantly mediated by alloreactive T cells present in the host which recognize donor alloantigens or xenoantigens. Immune tolerance is the acquired lack of specific immune responsiveness to an antigen to which an immune response would normally occur. Typically, to induce tolerance, there must be an exposure to a tolerizing antigen, which results in the death or functional inactivation of certain lymphocytes. This process generally accounts for tolerance to self antigens, or self-tolerance. Immunosuppressive agents are useful in prevention or reduction of undesired immune responses, e.g., in treating patients with autoimmune diseases or with allogeneic transplants. Conventional strategies for generating immunosuppression associated with an undesired immune response are based on broad-acting immunosuppressive drugs. Additionally, in order to maintain immunosuppression, immunosuppressant drug therapy is generally a life-long proposition. Unfortunately, the use of broad-acting immunosuppressants is associated with a risk of severe side effects, such as tumors, infections, nephrotoxicity and metabolic disorders. Accordingly, new immunosuppressant therapies would be beneficial.

### DESCRIPTION OF THE INVENTIONS AND THE PREFERRED EMBODIMENT

In one aspect, the current invention discloses a transdermal drug delivery system such as a transdermal patch to treat allergy comprising an antigen causing said condition and an immune activity enhancing agent. The antigen can be allergen, allergen or its fragment in form of its B cell antigen, T cell antigen in MHC-peptide complex form or the antigen peptide (or its derivative) of T cell antigen that can bind with MHC to form the MHC-peptide complex. Example of immune activity (or called immune function) enhancing agent can be selected from TLR agonist such as imiquimod and poly IC. The current invention also discloses a method to treat allergy or inhibit IgE induced reaction by inducing antigen specific immune tolerance and/or inducing the production of competing IgG against the antigen in a subject by administering to the subject a said transdermal drug delivery system on the skin.

The current invention further discloses methods and reagents to treat allergy by applying the combination of antigen causing allergy and immune activity enhancing agent/drug either as a physical mixture or as synthetic conjugate or as nano/micro particles or liposome to the object/patient in need at a therapeutically effective amount. The term nano/micro particle means the particle is in either nanometer or micrometer range of size (diameter). For example, the nano/micro particle can be in the size range of 50 nm˜100 um.

Examples of suitable immune activity (function) enhancing agent include pattern recognition receptor (PRR) ligands, RIG-I-Like receptor (RLR) ligands, Nod-Like receptor (NLR) ligands, C-Type Lectin Receptors (CLR) ligands, STING agonist, and Toll-like receptor ligands such as a TLR3 ligand, a TLR4 ligand, a TLR5 ligand, a TLR7/8 ligand, a TLR9 ligand, or a combination thereof. The immune function enhancing agent can be a vaccine adjuvant. Preferably the Toll-like receptor ligand is a Toll-like receptors (TLR) agonist. Examples of suitable immune activity enhancing agent (immunity boosting agent) include PRR Ligand, TLR3 Ligand, RLR Ligand, TLR4 Ligand, TLR5 Ligand, TLR7/8 Ligand, TLR9 Ligand, NOD2 Ligand, interleukin 12, tumor necrosis factor, interferon gamma (IFN7), immunomodulatory imide drugs (IMiDs such as thalidomide, lenalidomide and pomalidomide, Treg inhibitory agent such as inhibitory antibody against Treg or their combinations. Many of them are commercially available (e.g. those listed in catalogue from invivoGen Inc) and can be readily used for the current invention. Example includes imidazoquinoline family of TLR7/8 Ligands (e.g. imiquimod (R837), gardiquimod, resiquimod (R848), 3M-052, 3M-852, 3M-S-34240), CpG ODNs such as ODN 1826 and ODN 2216, TLR agonist including TLR peptide agonist disclosed in patent applications WO2018055060A1, WO2013120073A1, WO2016146143A1 and US20180133295A1 and their citations, synthetic analogs of dsRNA, such as poly IC (e.g. Poly ICLC, polyIC-Kanamycin, PolyI:PolyC12U), TLR4/5 Ligands such as Bacterial lipopolysaccharides (LPS, e.g. monophosphoryl lipid A), bacterial flagellin (e.g. Vibrio vulnificus flagellin B), Glucopyranosyl lipid A (GLA), TLR7 agonist Loxoribine or their derivatives/analogues, or their combinations. They can be in form of active drug, prodrug, liposome, emulsion, micelle, insoluble precipitate (e.g. in complex with condensing agent), conjugated to polymer drug carrier (e.g. dextran) or encapsulated in biodegradable micro particle/nano particle (e.g. those made of biodegradable polymer such as PLA, PLGA, PCL, PGA or PHB). The use and preparation of immune function enhancing agent encapsulated micro particle/nano particle or its prodrug are well known to the skilled in the art. Examples of them suitable for the current invention can be found in or adopted from U.S. patent application Ser. Nos. 13/560,955, 12/764,569, 12/788,266, publication in Vaccine. 2014 May 19; 32(24):2882-95, Science. 2015 Jun. 19; 348(6241): aaa8205 and Nat Commun. 2016; 7: 13193. And their related citations. In one example, PLGA-R837/ADU-S100 (R837 and ADU-S100 encapsulated in Poly Lactide-co-Glycolide particles) nanoparticle are prepared using o/w single-emulsion method. Briefly, R837 (TLR7 ligand) and STING agonist ADU-S100 are dissolved in DMSO at 2.5 mg/ml for each. A total of 50 μL above R837/ADU-S100 solution is added to 1 ml PLGA (5 mg/ml) dissolved in dichloromethane. Next the mixture is homogenized with 0.4 ml 5% w/v PVA solution for 10 min using ultrasonication. The o/w emulsion is added to 2.1 ml of a 5% w/v solution of PVA to evaporate the organic solvent for 4 h at room temperature. PLGA-R837/ADU-S100 nanoparticles are obtained after centrifugation at 3,500 g for 20 min. Combination of vaccine adjuvant (immune activity enhancing agent) and allergen can also be encapsulated together in micro/nano particles. For example, R837 or R848 or SB 11285 is dissolved in DMSO at 2.5 mg/ml. A pollen allergen extract is dissolved in DMSO at 5 mg/ml. 50 μl R837 or R848 or SB 11285 and 50 μl allergen solutions in DMSO are added to 1 ml mPEG-PLGA (10 mg/ml) dissolved in acetonitrile. Next, the mixture was dropwise added into 5 ml water containing 100 mg poly IC. After 1 h stirring and 12 h standing, the nanoparticles are obtained after centrifugation at 22,000 g for 5 min.

Besides the immune function enhancing agents listed above, other molecules that can activate/boost the function of immune system and immune cell such as APC, B cell and T cells 105 are also considered as immune function enhancing agents and can also be incorporated into the formulation. Suitable immune function activating/boosting molecule can be selected from granulocyte macrophage colony-stimulating factor (e.g. sargramostim or molgramostim), immunostimulatory monoclonal antibody (e.g. Anti-KIR antibody such as Lirilumab, antibody for CD137 such as Urelumab or Utomilumab, antibody against PD-1 and PD-L1, and other check point inhibitors used clinically), heparan sulfate (HS) mimetics such as PG545 (pixatimod, pINN), FMS-like tyrosine kinase 3 ligand (FLT3L), other pattern recognition receptor agonists besides poly IC, CpG and imiquimod, T-cell-tropic chemokines such as CCL2, CCL1, CCL22 and CCL17, B-cell chemoattractant such as CXCL13, Interferon gamma, type I IFN (e.g. IFN-α, IFN-beta), tumor necrosis factor (TNF)-beta, TNF-alpha, IL-1, Interleukin-2 (IL-2 such as 115 aldesleukin, teceleukin or bioleukin), interleukin-10 (IL-10), IL-12, IL-6, IL-24, IL-2, IL-18, IL-4, IL-5, L-6, IL-9 and IL-13 or their derivatives such as PEGylated derivative, CDId ligand, Vα14/Vβ8.2 T cell receptor ligand, iNKT agonist, α-galactosylceramide (α-GalCer), α-glucosylceramide (α-GlcCer), α-glucuronylceramide, α-galacturonylceramide, Isoglobotriosylceramide (iGb3), HS44, interleukin 12, antibody against OX 40, tumor necrosis factor, interferon gamma (IFNγ), immunomodulatory imide drugs (immune enhancing IMiDs such as thalidomide, lenalidomide and pomalidomide), Treg inhibitory agent such as inhibitory antibody against Treg (such as antibody against CD4, CD25, FOXP3 and TGF-β or its receptor) or their combinations. CD25 is more abundant in Treg, targeting CD25 provide inhibitory effect to Treg selectively over other cytotoxic T cells. They can be added to the formulation described here in at therapeutically effective amount to be used.

In some embodiments, the immune activity enhancing agents may comprise any of the agents provided herein. The immune activity enhancing agent can be a compound that directly provides the immune enhancing (e.g., activating) effect on APCs or it can be a compound that provides the immune enhancing effect indirectly (i.e., after being processed in some way after administration). Immune activity enhancing agents, therefore, include prodrug forms of any of the compounds provided herein. Different immune activity enhancing agent can be used as a mixture and be used in combination in the current invention.

Immune activity enhancing agents also include nucleic acids that encode the peptides, polypeptides or proteins provided herein that result in an immune enhancing (e.g. activating) immune response. In embodiments, therefore, the immune activity enhancing agent is a nucleic acid that encodes a peptide, polypeptide or protein that results in an immune enhancing (e.g. activating) immune response. The nucleic acid can be coupled to synthetic nanocarrier. The nucleic acid may be DNA or RNA, such as mRNA. In embodiments, the inventive compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector.

In some embodiments, the immune activity enhancing agent s provided herein are coupled to synthetic nanocarriers or microcarriers. In preferable embodiments, the immune activity enhancing agent is an element that is in addition to the material that makes up the structure of the synthetic nanocarrier or microcarrier. For example, in one embodiment, where the synthetic nanocarrier or microcarrier is made up of one or more polymers, the immune activity enhancing agent is a compound that is in addition and coupled to the one or more polymers. As another example, in one embodiment, where the synthetic nanocarrier or microcarrier is made up of one or more lipids, the immune activity enhancing agent is again in addition and coupled to the one or more lipids. In embodiments, such as where the material of the synthetic nanocarrier or microcarrier also results in an effect to prevent allergy, the immune activity enhancing agent is an element present in addition to the material of the synthetic nanocarrier or microcarrier that results in allergy prevention effect.

Selecta's publications disclose synthetic nanocarrier methods, and related compositions, comprising B cell and/or MHC Class II-restricted epitopes and immune activity suppressive agents in order to generate immune responses. In their disclosure, the antigen/epitope is conjugated to the nanocarrier and immune suppressive agent is coupled to the nanocarrier. An alternative method and composition are to use nano/micro particle having antigen/epitope causing allergy non-covalently adsorbed to its surface and immune activity enhancing agent encapsulated within. The nano/micro particles can be made of biodegradable materials such as PLGA. These kinds of nano/micro particles (e.g. 10 nm˜10 um of diameter in size) can be given to the patient in need as injection or inhaler or orally or applied topically to induce anti allergy effect. The encapsulation of immune activity enhancing agent is well known to the skilled in the art and can be adopted from related publications readily. The surface of the nano/micro particles can have charged groups such as amino or carboxyl group to increase the binding of antigen/epitope causing allergy to its surface; it can also have a hydrophobic surface to allow binding antigen/epitope via hydrophobic interaction; or the combination of them. Introducing charged groups to the surface can be done by using surface modification or using amine or carboxyl group containing molecules to prepared the nano/micro particles. The antigen/epitope causing allergy can also be conjugated with a lipid molecule such as fatty acid or cholesterol to increase its binding to nano/micro particles. The adsorption of antigen/epitope causing allergy to the nano/micro particle surface can be done by incubating antigen/epitope with the nano/micro particle (e.g. 4 degree overnight in aqueous solution buffer such as 1×PBS) and then removing the unbound antigen/epitope (e.g. washing the nano/micro particle with aqueous buffer several times, similar to the ELISA plate coating procedure). In one example, 50 nm˜200 nm size PLGA nano particle encapsulated with 10% by weight of imiquimod prepared. Next the PLGA nano particle is mixed with allergen OVA (10 mg/mL) at 4C overnight to generate the OVA (ovalbumin) coated particle. The particle is washed 3 times with PBS to remove unbound OVA. In another example, imiquimod is dissolved in DMSO at 50 mg/ml. A total of 50 μL imiquimod is added to 1 ml PLGA (5 mg/ml) dissolved in dichloromethane. Next the mixture is homogenized with 0.4 ml 5% OVA solution for 10 min using ultrasonication. The o/w emulsion is added to 2.1 ml of a 5% w/v solution of PVA to evaporate the organic solvent for 4 h at room temperature. OVA coated nano particles containing imiquimod are obtained after centrifugation at 3,500 g for 20 min. Additional washing step can be performed to obtain unbound OVA free particles. This OVA coated particle can be given to the target in need to induce OVA immune tolerance due to IgE to treat allergy against OVA, using the similar protocol described in the publications (e.g. those from Selecta Bio). The OVA can be replaced with other antigen/epitope molecule to induce corresponding immune activating to treat corresponding allergy. In another sample, lipophilic carboxylic acid or lipophilic amine or anionic detergent or cationic detergent (e.g. fatty acid such as caprylic acid, lauric acid; or cationic lipid such as DOTMA, DOTAP, cholesterylamine) can be added to the PLGA to prepare PLGA particle having surface charge. In one example, imiquimod is dissolved in DMSO at 50 mg/ml with lauric acid at 10 mg/mL. A total of 50 μL imiquimod/lauric acid is added to 1 ml PLGA (5 mg/ml PLGA) dissolved in dichloromethane. Next the mixture is homogenized with 0.1 ml 2% caprylic acid solution for 10 min using ultrasonication. The o/w emulsion is evaporated to remove the organic solvent for 4 h at room temperature. The resulting PLGA particle is washed 3 times with PBS and then incubated with OVA to prepare OVA bound particles. It can used to treat allergy to OVA. In one example, 10 mg˜100 mg of the particle can be injected to a patient with OVA intolerance bi-weekly for 3 times to induce OVA tolerance as subcutaneous or intravenous injection or intralymphatic injection.

Furthermore, antigen/epitope causing allergy can also be encapsulated within the nano/micro particle besides being conjugated or adsorbed to its surface. The preparation of antigen/epitope encapsulation is well known to the skilled in the art and can be adopted from related publications readily, e.g. using a double emulsion water/oil/water system.

US patent application 20130287729 A1 disclosed antigen-specific, tolerance-inducing microparticles and uses thereof. It disclosed a microparticle (0.5 μm-10.0 μm in size) for targeting an antigen-presenting immune cell of interest and for inducing antigen-specific immune tolerance, wherein the microparticle comprises an antigen and a therapeutic agent wherein the therapeutic agent is an immunomodulatory agent, an immunosuppressive tolerogenic agent, or an agent that recruits the antigen-presenting immune cell of interest, wherein the surface of the microparticle comprises a ligand that targets the antigen-presenting immune cell of interest and the microparticle is made of biodegradable material. A further improvement of this method and composition to treat allergy is to use micro particle or nano particle having the size of 50 nm˜5 um, preferably made of biodegradable materials and use immune activity enhancing agent instead of the immunosuppressive agent. The particle comprises an antigen causing allergy by encapsulation or coating or both. In some embodiments, the surface of the nano/micro particle is coated with Fc portion of an antibody or a full antibody with its Fc portion facing outside. This will bind with the FcR to facilitate APC uptake. In other embodiments, the surface of the nano/micro particle needs not to have a ligand that targets the antigen-presenting immune cell. In some embodiments, it can have antigen/epitope causing allergy coated on its surface. The inner part of the nano/micro particle contains immune activity enhancing agent listed in the current application and optionally antigen/epitope causing allergy, e.g. by encapsulation. The preparation method is well known to the skilled in the art and can be adopted from related publications readily. For example, 5 mg˜50 mg of the above particle containing gluten and poly IC can be injected to a patient with gluten intolerance weekly for 3 times to induce gluten tolerance as subcutaneous or intravenous injection or intralymphatic injection.

US patent application 20160338953 A1 disclosed a liposome-based immunotherapy. It provided a liposome encapsulating an autoantigen, wherein the liposome has a size comprised from 500 to 15000 nm and the liposome membrane comprises phosphatydilserine (PS) in an amount comprised from 10 to 40% by weight with respect to the total membrane liposomal composition. Pharmaceutical or veterinary compositions comprising a therapeutically effective amount of said liposome were also provided. Further, it provided liposomes and pharmaceutical or veterinary compositions as defined above for use as a medicament, particularly for the treatment of autoimmune diseases. Finally, it provided liposomes and pharmaceutical or veterinary compositions as defined above for use in the restoration of tolerance to self in a patient suffering from an autoimmune disease. The current invention also discloses antigen-specific liposome for allergy treatment and uses thereof. The liposome contains immune activity enhancing agent listed in the current application (and optionally antigen/epitope molecule causing allergy) inside by encapsulation. Optionally the surface of the liposome can also have allergy causing antigen/epitope coated. It can be given to the patient in need as injection or inhaler or orally or nasal drop or applied topically to induce immune tolerance for allergen to treat allegy. The lipid used for liposome can include but not limited to phosphatydilserine at 10 to 40% by weight of the membrane. It can also use non-phosphatydilserine lipid to prepare the membrane. The antigen/epitope causing allergy can also be conjugated with a lipid type molecule such as fatty acid or phospholipid or cholesterol derivative to allow it to be inserted to the liposome membrane. Suitable liposome can have a size between 50 nm˜20 um. The preparation method and the protocol of its use are well known to the skilled in the art and can be adopted from related publications readily such as those in US20160338953. Example of the lipid molecule suitable for the current invention to prepare liposome includes but is not limited to phospholipid glycerolipid, glycerophospholipid, sphingolipid, ceramide, glycerophosphoethanolamine, sterol or steroid. These lipid molecules can also be used to prepare the allergy causing antigen/epitope-lipid conjugate. Membrane anchoring peptide-antigen/epitope conjugate can also be used instead of antigen/epitope-lipid conjugate.

The current invention discloses methods and regents to treat allergy by applying the mixture of antigen causing allergy and immune activity enhancing agent topically to the object/patient in need. It can also contain anti-allergy drug such as Antihistamines, Corticosteroids, Mast cell stabilizers, and Leukotriene inhibitor. The addition of these anti-allergy drugs can prevent the allergy reaction induced by giving the allergen to the patient. The method and the said mixture/formulation can be used to induce the generation of anti-allergen IgG antibody to compete the endogenous IgE which will generate allergy reaction; therefore it will induce immune tolerance for the allergen. Examples of the formulation suitable for the current application include solid form such as powder, gel, lotion, ointment, solution, spray, suppository, lozenge, tablet and patch that can be inhaled, injected or taken orally or topically applied to the skin or mucosa. The term topical drug delivery include drug delivery route other than injection. It includes applying drug to skin or mucosa. It includes intranasal delivery, rectal delivery, nasal mucosa delivery, sublingual delivery and oral mucosa delivery. It can be a mucosa adhesive tablet or mucosa adhesive lozenge, which contains mucosa adhesive agent to attach it to mucosa such as oral mucosa. The immune activity enhancing agent can be in the form of active agent, prodrug form, micro particle or nano particle form or liposome form. The antigen causing allergy can be either B cell antigen/epitope or T cell antigen/epitope (e.g. MHC-peptide complex or conjugate; or the antigen fragment such as peptide that can bind with MHC) or their combination. The combination can be either B cell antigen/epitope with T cell antigen/epitope; or the combination of several different B cell antigen/epitope and/or several different T cell antigen/epitope targeting the same disease or different diseases. The use of peptide antigen (T cell epitope) that can bind with MHC to form MHC-peptide complex in vivo (T cell antigen) instead of the peptide-MHC complex reduce the size and molecular weight, therefore improve the transdermal delivery. The use of peptide antigen having single epitope domain can reduce the risk of activating mast cells by not cross linking the IgE on cell surface, therefore provide better safety yet still be cable to induce immune tolerance. The allergy causing antigen (allergen) used in the current invention can be either full allergen or its fragment such as its epitope, or their combination. Examples of them can be found in the current application and related publications and patent applications.

A mixture of allergy causing antigen and immune activity enhancing agent can be a physical mixture. A physical mixture means that the mixture of antigen and immune activity enhancing agent are simply mechanically mixed (e.g. by stirring or blending) together in their original form (e.g. liquid or solid form such as powder or particles) without any additional process (e.g. by mixing them in their original form together), or further size reducing process is applied after the mechanical mixing (e.g. crashing, grinding, mulling or homogenizing), or dispersed or dissolved separately in same or different type of liquid and then mix, or co-dispersed in liquid, or co-dissolved in solvent (e.g. water), and optional drying process (e.g. spray drying or lyophilization) can be applied with optional further size reducing process.

In some embodiments, the method is to use a patch containing both allergen or its fragment and immune enhancing drug (the drug listed above such as imiquimod or poly IC). It can also contain anti-allergy drug such as Antihistamines, Corticosteroids, Mast cell stabilizers, and Leukotriene inhibitor.

The transdermal or transmucosal delivery of both allergy causing antigen and immune enhancing drug will induce immune tolerance for allergen via DC cells in the skin or mucosa. The skin may be exfoliated to remove stratum corneum layer to increase drug delivery or using a micro needle system. This would be a much easier strategy for food allergy than injection. The skin may be intact or may be exfoliated to remove stratum corneum layer to increase drug delivery. Micro needle system can also be used to the skin. The micro needle in the micro needle system can be made of bio degradable material such as PLGA encapsulating antigen and immune activity enhancing agent. Alternatively, a bio degradable implant encapsulating allergy causing antigen and immune activity enhancing agent can be used instead of transdermal or transmucosal delivery system for allergy treatment and prevention. The size of the implant can be bigger than 10 um in diameter, preferably >100 um, if the implant is a macro particle. The macro particle can be in the size range of 100 um-10 mm. The particles can be made of biodegradable materials such as PLGA. The implant can also be non-sphere shape. For example, a 2 mm (length)×0.3 mm (diameter) rod made with PLGA containing 3 mg imiquimod and 1 mg gliadin or a 5 mm (length)×2 mm (diameter) rod made with PLGA containing 1 mg imiquimod and 100 mg gliadin can be used as an implant underneath the skin to treat gluten intolerance. Other implant format including non-degradable device can also be used such as NanoPortal Capsule from Nanoprecision Medical and Medici Drug Delivery System from Intarcia, as long as they can deliver the antigen and immune activity enhancing agent simultaneously. In some embodiments, sustained release implant such as NanoPortal Capsule from Nanoprecision Medical and Medici Drug Delivery System from Intarcia can contain allergen only without the need of immune enhancing agent. Current allergen injection to treat allergy need to be injected very frequently, using the implant to provide sustained release of allergen will reduce the implantation (e.g. one implant every month or very 3 months) frequency and will be more patient friendly. The dose of the allergen in the first implant is low and increase gradually in the later implant to ensure the safety. The implant can also contain therapeutically effective amount (e.g. the dose currently used in clinic) of anti-allergy drug such as Antihistamines, Corticosteroids, Mast cell stabilizers, and Leukotriene inhibitor. In some embodiments, the implant can release a daily dose of antigen same as the dose of the current antigen injection used for allergy prevention/treatment and the release can last several weeks to several months. Preferably the released daily dose is able to induce tolerance to allergen but not cause severe allergy reaction such as severe immediate hypersensitivity reactions. For example, an implant contains 0.3-3 mg allergen which allows the release of 0.1 ug˜0.1 mg allergen daily for 30 days and the allergen can be peanut protein or egg white protein or pollen extract. If severe allergy reaction is observed, the implant can be removed from the patient to increase its safety.

DBV Technologies and other groups (e.g. those described in Epicutaneous Immunotherapy for Aeroallergen and Food Allergy DOI: 10.1007/s40521-013-0003-8) are using skin patch containing allergen to treat allergy by inducing tolerance for the antigen (allergen). The topically patch or other formulation can be readily adopted for the current application. For example, the topical applied formulation such as patch described in U.S. Ser. No. 15/135,914, U.S. Pat. No. 6,676,961, U.S. Ser. No. 15/111,204, U.S. Pat. No. 8,932,596B2, U.S. Ser. No. 15/184,933A1 and U.S. Pat. No. 8,202,533B2 can be adopted for the current application by adding additional immune enhancing drug (e.g. 0.1 mg-20 mg of imiquimod or poly IC or their directives or prodrug) into the patch or those commercial available patch (e.g. VIASKIN® MILK and VIASKIN® PEANUT). The administration method can be essentially the same as the prior arts except the patch contains immune activity enhancing agents. Additional transdermal enhancer (e.g. DMSO, Azone, fatty acid, hyaluronic acid and etc, which can be found in the publication readily as well as their suitable amount) can be added to the patch or applied to the skin before applying the patch. Example of transdermal enhancing agent can be added include DMSO (e.g. 10˜300 mg/patch), azone (e.g. 1%˜10% of total drug weight), surfactant, fatty acid (e.g. 1%˜10% oleic acid). The stratum corneum of skin can be removed with exfoliation or other means to enhance the transdermal delivery. In one example, the patch contains 500 ug˜10 mg gluten (e.g. G5004 Gluten from wheat, Sigma) and 0.1 mg˜10 mg of imiquimod or 1 mg˜50 mg R848. For example, allergy causing antigen such as gluten and immune activity enhancing agent such as imiquimod and/or poly IC can be in powder form, which can be simply mixed together physically, they can also be co-dissolved and then dried and then placed in the patch.

For example, 10 mg gluten powder and 1 mg of imiquimod powder are blended and then homogenized with a grinder, and then applied to the surface of the skin contact side of a 5×5 cm² dermal patch. In another example, 10 mg gluten and 100 mg of poly IC are mixed in 10 mL water containing 30 mg sucrose vigorously for 10 min and then lyophilized, and then the dry mixture is applied to the surface of the skin contact side of a 5×5 cm² dermal patch. In another example, 10 mg gluten, 10 mg of STING agonist MK-1454 and 50 mg of CpG ODN are dissolved in 5 mL 25% EtOH water solution and then vacuum dried, and then the dry mixture is placed to the surface of the skin contact side of a 3×3 cm² dermal patch. In another example, 10 mg gluten and 5 mg of imiquimod are dissolved in 5 mL 1% SDS water solution and then vacuum dried, and then the dry mixture is placed to the surface of the skin contact side of a 3×2 cm² dermal patch. In another example, the 10×10 cm² patch contains 5 mg gluten (e.g. G5004 Gluten from wheat, Sigma) and 5 mg of imiquimod or 50 mg 3M-052 and optionally additional 30 mg azone. In another example, the patch contains 5 mg gluten (e.g. G5004 Gluten from wheat, Sigma) and 100 mg of sialic acid or sialic acid-cholesterol conjugate and 10 mg poly IC. This can be used to induce gluten tolerance and treat gluten intolerance. The gluten can be replaced with gliadin instead. In embodiments, the gluten or gliadin containing patch can be applied daily to forearm 8 hours a day for 1-5 weeks. The gluten in the above examples can be replaced with egg white protein such as 5-10 mg of ovomucoid (Gal d 1) or 5-10 mg ovalbumin (Gal d 2) or their combination with optional 5-10 mg ovotransferrin (Gal d 3) and 5-10 mg lysozyme (Gal d 4) to treat egg white allergy. In another example, the antigen is peanut antigen ara h2 200 ug and 2 mg of imiquimod is in the patch to treat peanut allergy. In one example, peanut antigen ara h2 200 ug, 2 mg of imiquimod and 50 mg sucrose is dissolved in water and then lyophilized and then placed in the patch. In one example, peanut antigen ara h2 200 ug, 2 mg of imiquimod, 50 mg SDS and 50 mg sucrose is dissolved in water and then lyophilized and then placed in a 5×5 cm² patch. In one example, peanut antigen ara h2 200 ug, 2 mg of imiquimod, 100 mg DMSO and 50 mg sucrose is dissolved in water and then lyophilized and then placed in the patch. In another example, the nasal spray or nasal drop contains 1 mg gluten (e.g. G5004 from Sigma, Gluten from wheat) and 1 mg of imiquimod or 1 mg poly IC in a suitable form for each spray or every 3 drops, viscosity enhancing agent can be added, such as hyaluronic acid or carbomer. In another example, the sublingual lozenge contains 50 mg gluten (e.g. G5004 from Sigma, Gluten from wheat) and 1 mg of imiquimod or 20 mg poly IC. In another example, a gel contains 50 mg gluten (e.g. G5004 Gluten from wheat, Sigma) and 2 mg of imiquimod or 20 mg poly IC in each iml of gel. The immune activity enhancing agent drug or both the immune activity enhancing agent drug and the allergy causing antigen can be either in the form of powder or gel or semi liquid or in the form of liposome (e.g. 100 nm˜5 um diameter) or in a nano/micro particle (e.g. 100 nm˜um) or being conjugated to a dendrimer or linear polymer (e.g. couple to poly acrylic acid or poly Sialic acid via ester bond to form a polymer based prodrug with MW=5K˜500K).

Other pharmaceutically acceptable amount of antigen causing allergy and immune activity enhancing agent can also be used in the patch, as long as it can produce satisfactory biological and therapeutical (e.g. immune tolerance) effect, which can be determined experimentally by screening and testing with well-known protocol and methods.

Other allergen such as dust mite extract, pollen extract can also be used instead of peanut protein.

The allergy causing antigen can be either B cell antigen/epitope or T cell antigen/epitope (e.g. MHC-peptide complex or conjugate; or the antigen fragment such as peptide that can bind with MHC) or their combination. Examples of them can be found in the current application, previous applications by the same inventor and related publications and patent applications.

A skin patch (also called transdermal patch) is a medicated adhesive patch or attachable patch that is placed on the skin to deliver a specific dose of medication through the skin and into the bloodstream. A wide variety of pharmaceuticals are now available in transdermal patch form.

There are several main types of skin/transdermal patches. The Single-layer Drug-in-Adhesive type is that the adhesive layer of this system also contains the drug. In this type of patch the adhesive layer not only serves to adhere the various layers together, along with the entire system to the skin, but is also responsible for the releasing of the drug. The adhesive layer is surrounded by a temporary liner and a backing. The Multi-layer Drug-in-Adhesive type is the multi-layer drug-in-adhesive patch is similar to the single-layer system; the multi-layer system is different, however, in that it adds another layer of drug-in-adhesive, usually separated by a membrane (but not in all cases). One of the layers is for immediate release of the drug and other layer is for control release of drug from the reservoir. This patch also has a temporary liner-layer and a permanent backing. The drug release from this depends on membrane permeability and diffusion of drug molecules. The Reservoir type is unlike the single-layer and multi-layer drug-in-adhesive systems, the reservoir transdermal system has a separate drug layer. The drug layer can be a liquid or gel or powder compartment containing a drug solution or suspension or powder separated by the adhesive layer. This patch is also backed by the backing layer. In this type of system the rate of release is zero order. The Matrix type has a drug layer of a solid or semisolid matrix containing a drug solution or suspension or solid layer such as powder or film. The adhesive layer in this patch surrounds the drug layer, partially overlaying it. In some embodiments, the reservoir type and the matrix type can be used for current invention.

In one example, allergy causing antigen (either full allergen or its fragment or epitope) and immune activity enhancing agent loaded matrix-type transdermal patch is prepared by using solvent casting method. A petri dish with a total area of 50 cm² is used. Antigen and immune activity enhancing agent are dissolved in 5 mL of water, methanol (1:1) solution and mixed until clear solution is obtained. 200 mg polyethylene glycol 400 is used as plasticizer and optional 100 mg propylene glycol or oleic acid or tween 80 is used as permeation enhancer, together with 100 mg sucrose they are added to the antigen/immune activity enhancing agent solution. The resulted uniform solution is cast on the petri dish, which is lubricated with glycerin and lyophilized or dried at room temperature for 24 h. Next the dried patch is placed on a cellulose acetate membrane used as backing membrane. In another example, weighed amount of PVA (2.5% w/v) is added to a distilled water and a homogenous solution is made by constant stirring and intermittent heating at 60° C. for a few seconds and poured into glass molds already wrapped with aluminium foil around open ends and are kept for drying at 60° C. for 6 h, forming a smooth, uniform, and transparent backing membrane. Backing membrane is used as a support for antigen and immune activity enhancing agent containing matrix.

In some embodiments, the skin patch device used in the method of the invention preferably comprises a backing, the periphery of said backing being adapted to create with the skin a hermetically closed chamber. This backing bears on its skin facing side within the chamber the composition used to decrease the skin reactivity. Preferably, the periphery of the backing has adhesive properties and forms an airtight joint to create with the skin a hermetically closed chamber.

In a particular embodiment, the composition allergens and immune activity enhancing agent s are maintained on the backing by means of electrostatic and/or Van der Waals forces. This embodiment is particularly suited where the composition allergens are in solid or dry form (e.g., particles), although it may also be used, indirectly, where the allergens are in a liquid form. Within the context of the present invention, the term “electrostatic force” generally designates any non-covalent force involving electric charges. The term Van der Waals forces designates non-covalent forces created between the surface of the backing and the solid allergen and may be of three kinds: permanent dipoles forces, induced dipoles forces, and London-Van der Waals forces. Electrostatic forces and Van der Waals forces may act separately or together. In this respect, in a preferred embodiment, the patch device comprises an electrostatic backing. As used herein, the expression “electrostatic backing” denotes any backing made of a material capable of accumulating electrostatic charges and/or generating Van der Waals forces, for example, by rubbing, heating or ionization, and of conserving such charges. The electrostatic backing typically includes a surface with space charges, which may be dispersed uniformly or not. The charges that appear on one side or the other of the surface of the backing may be positive or negative, depending on the material constituting said backing, and on the method used to create the charges. In all cases, the positive or negative charges distributed over the surface of the backing cause forces of attraction on conducting or non-conducting materials, thereby allowing to maintain the allergen and immune activity enhancing agent. The particles also may be ionized, thereby causing the same type of electrostatic forces of attraction between the particles and the backing. Examples of materials suitable to provide electrostatic backings are glass or a polymer chosen from the group comprising cellulose plastics (CA, CP), polyethylene (PE), polyethylen terephtalate (PET), polyvinyl chlorides (PVCs), polypropylenes, polystyrenes, polycarbonates, polyacrylics, in particular poly(methyl methacrylate) (PMMA) and fluoropolymers (PTFE for example). The foregoing list is in no way limiting.

The back of the backing may be covered with a label which may be peeled off just before application. This label makes it possible, for instance, to store the composition allergen in the dark when the backing is at least partially translucent. The intensity of the force between a surface and a particle can be enhanced or lowered by the presence of a thin water film due to the presence of moisture. Generally, the patch is made and kept in a dry place. The moisture shall be low enough to allow the active ingredient to be conserved. The moisture rate can be regulated in order to get the maximum adhesion forces. As discussed above, the use of an electrostatic backing is particularly advantageous where the allergen is in a dry form, e.g., in the form of particles. Furthermore, the particle size may be adjusted by the skilled person to improve the efficiency of electrostatic and/or Van der Waals forces, to maintain particles on the support.

In a specific embodiment, the patch comprises a polymeric or metal or metal coated polymeric backing and the particles of composition allergens are maintained on the backing essentially by means of Van der Waals forces. Preferably, to maintain particles on the support by Van der Waals forces, the average size of the particles is lower than 60 micrometer. In another embodiment, the allergens are maintained on the backing by means of an adhesive coating on the backing. The backing can be completely covered with adhesive material or only in part. Different occlusive backings can be used such as polyethylene or PET films coated with aluminum, or PE, PVC, or PET foams with an adhesive layer (acrylic, silicone, etc.). Examples of patch devices for use in the present invention are disclosed in U.S. patent application Ser. No. 11/915,926 or U.S. Pat. No. 7,635,488.

Other examples are disclosed in patent application U.S. Ser. No. 13/230,689, which also discloses a spray-drying process to load the substance in particulate form on the backing of a patch device. An electrospray device uses high voltage to disperse a liquid in the fine aerosol. Allergens and immune activity enhancing agent s dissolved in a solvent are then pulverized on the patch backing where the solvent evaporates, leaving allergens and immune activity enhancing agent s in particles form. The solvent may be, for instance, water or ethanol, according to the desired evaporation time. Other solvents may be chosen by the skilled person. This type of process to apply substances on patch backing allows nano-sized and mono-sized particles with a regular and uniform repartition of particles on the backing. This technique is adapted to any type of patch such as patch with backing comprising insulating polymer, doped polymer or polymer recovered with conductive layer. Preferably, the backing comprises a conductive material.

In another embodiment, the periphery of the backing is covered with a dry hydrophilic polymer, capable of forming an adhesive hydrogel film by contact with the moistured skin (as described in U.S. Ser. No. 12/680,893). In this embodiment, the skin has to be moistured before the application of the patch. When the hydrogel comes into contact with the moistured skin, the polymer particles absorb the liquid and become adhesive, thereby creating a hermetically closed chamber when the patch is applied on the skin. Examples of such hydrogels include polyvinylpyrolidone, polyacrylate of Na, copolymer ether methyl vinyl and maleic anhydride.

In another particular embodiment, the liquid composition allergen and immune activity enhancing agent is held on the support of the patch in a reservoir of absorbent material. The composition may consist in an allergen+immune activity enhancing agent solution or in a dispersion of the mixture, for example in glycerine. The adsorbent material can be made, for example, of cellulose acetate.

The backing may be rigid or flexible, may or may not be hydrophilic, and may or may not be translucent, depending on the constituent material. In the case of glass, the support may be made break-resistant by bonding a sheet of plastic to the glass. In one embodiment, the backing of the patch contains a transparent zone allowing directly observing and controlling the inflammatory reaction, without necessarily having to remove the patch. Suitable transparent materials include polyethylene film, polyester (polyethylene-terephtalate) film, polycarbonate and every transparent or translucent biocompatible film or material.

Current invention also discloses methods and regents to treat or prevent allergy by applying the mixture of allergy causing antigen and said immune activity enhancing agent/drug as injection to the object/patient in need. The injection can be given as either subcutaneous injection or intramuscular injections or intradermal injections. The injection can contain a viscosity enhancing agent to increase its viscosity when it is being injected, which acts as a sustained release formulation of both antigen and immune activity enhancing agent Allergy causing antigen and immune activity enhancing agent can be either in free molecule form or in nano/micro particle from including liposome form. In certain embodiments, the injection has a viscosity greater than 10,000 cps at room temperature. In certain embodiments, the injection has a viscosity greater than 100,000 cps at room temperature. In certain embodiments, the injection has a viscosity greater than 5,000,000 cps at room temperature. In certain embodiments, the injection has a viscosity of 11,000,000 cps at room temperature. Example of the viscosity enhancing agent can be found readily from known pharmaceutical acceptable excipient such as hyaluronic acid (linear or crosslinked), starch and carbomer. In some embodiments, the viscosity enhancing agent is biodegradable. In one example, a viscous injection contains 5 ug-5 mg/mL gluten (e.g. G5004 Gluten from wheat, Sigma) and 50 ug-5 mg/mL of imiquimod or 500 ug-50 mg/mL poly IC and suitable amount of hyaluronic acid (e.g. 50 mg/mL linear or cross linked hyaluronic acid) to reach a viscosity of 5,000,000 cps. In one example, a viscous injection contains 10 ug mg/mL gluten (e.g. G5004 Gluten from wheat, Sigma) and 50 ug mg/mL of imiquimod or 100 ug/mL poly IC and suitable amount of hyaluronic acid (e.g. 50 mg/mL cross linked hyaluronic acid) to reach a viscosity of 10,000,000 cps. The injection formulation can also be a thermal phase changing formulation. Thermal phase changing formulation is a formulation that change its phase from liquid at low temperature or room temperature (25° C.) to semisolid/gel when temperature increases to body temperature (37° C.), which can use poloxamer as excipient. A thermal phase changing injectable formulation containing both antigen and immune activity enhancing agent can be given as either subcutaneous injection or intramuscular injections or intradermal injections to induce antigen specific immune tolerance and treat corresponding auto immune diseases or allergy. It has low viscosity at low or room temperature but high viscosity at body temperature. The preparation of this kind of thermal phase changing injectable formulation can be adopted from related publications readily by the skilled in the art. For example, a composition of a thermal phase changing injectable formulation is 15 mg/mL gluten (e.g. G5004 Gluten from wheat, Sigma) and 3 mg/mL imiquimod in 25% (w/w) Poloxamer-407 pH=7 solution, which can be injected to a patient with gluten intolerance 1 mL bi-weekly for 3 times to treat allergy to gluten as subcutaneous or intravenous injection or intralymphatic injection. The gluten in the above examples can be replaced with egg white protein such as 5-10 mg of ovomucoid (Gal d 1) or 5-10 mg ovalbumin (Gal d 2) or their combination with optional 5-10 mg ovotransferrin (Gal d 3) and 5-10 mg lysozyme (Gal d 4) to treat egg allergy. It can also contain anti-allergy drug such as Antihistamines, Corticosteroids, Mast cell stabilizers, and Leukotriene inhibitor. The amount of anti-allergy drug added can be the same as those currently used in anti allergy treatment. The addition of these anti-allergy drugs can prevent the allergy reaction induced by giving the allergen to the patient. Furthermore, those reagents and formulations can also be injected into lymph node instead for allergy treatment. Intralymphatic allergen administration is known and the same procedure can be readily adopted for the current invention. In some embodiments, the amount of the reagent or formulation injected into lymph node is between 0.001 mg˜10 mg allergen with injection volume between 0.1 ml to 1 ml per lymph node once weekly or monthly for 3 weeks or 3 months.

The immune activity enhancing agent can also be conjugated to carbohydrate polymer or other bio compatible polymer (e.g. dextran or heparin or hyaluronic acid or poly peptide) to form prodrug as described in U.S. patent application Ser. Nos. 15/723,173; 16/380,951 and 16/029,594. The novel prodrugs can be in the form of carbohydrate (or other polymer) drug conjugate in which the drug can be conjugated to the carbohydrate (or other polymer) with cleavable linkage. More than one drugs can be conjugated to the polymer backbone. Suitable carbohydrate includes sialic acid containing polymer, hyaluronic acid, chondroitin sulfate, dextran, carboxyl dextran, cellulose, carboxyl cellulose and their derivatives. In some embodiments, preferably the carbohydrate is selected from sialic acid containing polymer, hyaluronic acid, starch, dextran and chondroitin sulfate. The sialic acid containing polymer suitable for the current invention include poly sialic acid formed by sialic acid monomer connected with α2,3 or α2,6 or α2,8 or α2,9 linkage or their combination. It also includes graft polymer or branched polymer containing sialic acid. It can also be a linear polymer backbone (e.g dextran or synthetic polymer such as PVA, PAA). Furthermore, the immune enhancing drug can also be directly conjugated to antigen or conjugated to the antigen via a linker or carrier. The conjugate can also be used in the patch. The carrier can be a polymer. For example, the poly sialic acid-rapamycin in FIG. 8 of U.S. application Ser. No. 15/723,173 can be used to conjugate to the protein's lysine with EDC coupling (e.g. gluten or antibody drug or gliadin or is peanut antigen protein ara h2) and be used in the patch (e.g. 100 ug˜15 mg) instead of the mixture of antigen and drug.

The allergy causing antigen can be conjugated to a carrier to from a multimer. The allergy causing antigen and immune enhancing drug can also be conjugated together. They can be in the form of linear polymer, micro particle, nano particle, liposome, implant or a transdermal drug delivery system such as a transdermal patch. A carrier system can be used for the previous and current applications to construct the conjugate. For example, the liposome or microparticle or nanoparticle can be used as a carrier. The antigen can be immobilized on the surface of the liposome or particles and the immune enhancing agent can be either encapsulated inside or co-immobilized on the surface of liposome or particles. The carrier can also be a linear or branched polymer such as dextran, hyaluronic acid, heparin, chondroitin sulfate and poly peptide. Both allergy causing antigen and immune enhancing agent can be conjugated to the polymer. They can be given to the subject in need to treat allergy by administering to the subject said conjugate (e.g. subcutaneous or intravenous injection or applied to the skin such as the skin of upper arm). Additional details can be found in the previous disclosures.

When liposome is used, either the drug or both the antigen and immune enhancing drug can be encapsulated in the liposome. Besides being applied topically, the mixture or conjugate can also be injected or taken orally to induce immune tolerance to treat allergy.

The topical formulation or implant or injection can contain either antigen+drug or antigen-drug conjugate or encapsulated antigen/drug (e.g. in microsphere or liposome) or their combinations. The antigen can be either in the form of crude antigen (e.g. peanut extract, gluten, pollen extract, dust mite extract) or purified antigen (e.g. peanut antigen protein ara h2, gliadin) or antigen-drug conjugate or encapsulated antigen (e.g. in microsphere or liposome) or their mixture.

Another format suitable for the current application is to use microsphere. The term microsphere include particles from nano meter size to micrometers (e.g. 50 nm˜50 um in diameter). Preferably the microsphere is bio degradable (e.g. made of biodegradable polymer such as poly(lactidecoglycolide)(PLGA)), the microsphere can further encapsulate immune suppressive drug such as imiquimod (e.g. 1%˜80% weight of the microsphere).

For example, the microsphere can be biodegradable synthetic polymer such as PLGA. Immune enhancing drug such as imiquimod (e.g. 1%˜80% weight of the microsphere) is encapsulated. The size of the microsphere is 3 um or 300 nm. Antigen is also conjugated to the surface of the microsphere directly or with a linker. The antigen can also be encapsulated in the microsphere as well. Alternatively, the drug (immune activity enhancing agent) can be conjugated to the surface of the microsphere instead of being encapsulated. Examples of microsphere suitable for the current application can be readily adopted from the disclosure in the publications such as those in U.S. patent application Ser. Nos. 13/880,778, 14/934,135, CA 2910579, U.S. Ser. No. 13/084,662 and U.S. Pat. No. 8,652,487 and other patent application filed by Selecta Biosciences. It can be used to treat allergy, which can be either injected or implanted (being encapsulated inside the implant) or applied topically to the patient. The pharmaceutically acceptable amount of these types of conjugate can also be used, as long as it can produce satisfactory therapeutical (e.g. immune tolerance) effect, which can be determined experimentally by screening and testing with well-known protocol.

Another format suitable for the current application is to use polymer carrier conjugated with allergen and immune activity enhancing agent. The polymer is conjugated with multiple antigen (e.g. 1-100), and multiple copies of immune activity enhancing agent (e.g. 5˜500 molecules).

In some embodiments, the topical formulations contain 0.1˜10 mg allergen, 0.1˜50 mg TLR7/8 Ligands (e.g. imiquimod or gardiquimod or resiquimod), 1˜50 mg TLR3/RLR Ligands (e.g. dsRNA such as poly IC or polyICLC), 1˜50 mg TLR9 Ligands (e.g. CpG ODNs such as ODN 1826 or ODN 2216) each patch or each mL of gel or lotion or liquid. Transdermal enhancing agent can be added to it as well such as DMSO, azone (e.g. 1%˜10/6), surfactant, fatty acid (e.g. 1%˜10% oleic acid). In one example, the formulations contain 5 mg/mL gluten, 5 mg/mL imiquimod, 5 mg/mL poly IC, 5 mg/mL class A CpG ODN 2216, 20 mg/mL SDS in 1×PBS and 5% sucrose and then being lyophilized. The lyophilized powder can be used to prepare a skin patch and attached to the skin at 10˜500 mg powder/patch. In another example, 10˜100 mg egg white powder, 5-50 mg of imiquimod, 5-50 mg of poly IC and 5-50 mg of azone is mixed together and added to a Viaskin® like dermal patch. It can be applied to the skin twice every week for 2 weeks, each time for 2 day and then applied for 2 days as a booster after 1 month and 3 month to generate egg tolerance. In another example, 10 mg peanut protein, 20 mg of imiquimod, 50 mg of poly IC and 100 mg of DMSO is mixed together and added within a Viaskin® like device. It can be applied to the skin twice every week for 2 weeks, each time for a and then applied for 2 day after 1 month and 3 month to generate peanut tolerance. In some embodiments, the topical formulations contain 0.1˜100 mg antigen, 0.1˜50 mg TLR agonist in each mL of gel or lotion or liquid; transdermal enhancing agent can be added to it as well such as DMSO, azone (e.g. 1%˜10%), surfactant, fatty acid (e.g. 1%˜10% oleic acid).

The formulation can also be an oral formulation such as a tablet or capsule containing the mixture of allergy causing antigen and immune enhancing agent. It can be the same as those used by Aimmune's oral formulation (e.g. its AR101 for peanut allergy) except additional immune enhancing agent is added. Viscosity enhancing agent can also be incorporated similar to those described above. It can also contain therapeutically effective amount (e.g. the dose currently used in clinic) of anti-allergy drug such as Antihistamines, Corticosteroids, Mast cell stabilizers, and Leukotriene inhibitor as described above. The addition of these anti-allergy drugs can prevent the allergy reaction induced by giving the allergen to the patient. In one embodiment, a formulation is an enteric capsule containing 0.1 mg-10 mg peanut protein, 10 mg imiquimod, 10 mg carbomer 940 and 10 mg Cetirizine. At later stage treatments, the peanut protein amount is increased as those used in AR101. In some embodiments, the immune enhancing agent can be replaced with silica acid or poly sialic acid or sialic acid polymer or siglec ligand or their derivatives (e.g. 50˜500 mg/capsule). Sialic acid polymer contains either α2,3 or α2,6 or α2,8 sialoside or sialic acid or their derivatives (e.g. those described in J Immunol. 2006 Sep. 1; 177(5):2994-3003, U.S. Pat. Nos. 9,522,183 and 8,357,671) that can bind with Siglec. The oligo/poly sialic acid with α2,8 linkage backbone itself is also a sialic acid polymer. The sialic acid polymer can also contain the mixture of different sialoside, sialic acid and/or their derivatives on its backbone. The liposome having sialic acid or sialoside attached on its surface can also be regarded as a sialic acid rich polymer (e.g. those described in U.S. Pat. No. 9,522,183). There are many sialic acid/siglec ligand and its polymer form suitable for the current application can be readily found in the literature, for example, those described in J Immunol. 2006 Sep. 1; 177(5):2994-3003, Nat Chem Biol. 2014 January; 10(1):69-75, J Am Chem Soc. 2013 Dec. 11; 135(49):18280-18283, J Immunol. 2014 Nov. 1; 193(9):4312-21, J Allergy Clin Immunol. 2017 January; 139(1):366-369.e2, Angew Chem Int Ed Engl. 2015 Dec. 21; 54(52):15782-8, Proc Natl Acad Sci USA. 2009 Feb. 24; 106(8):2500-5, J Exp Med. 2010 Jan. 18; 207(1):173-87, J Immunol. 2013 Aug. 15; 191(4):1724-31, Proc Natl Acad Sci USA. 2016 Sep. 13; 113(37):10304-9, J Clin Invest. 2013 July; 123(7):3074-83, Proc Natl Acad Sci USA. 2016 Mar. 22; 113(12):3329-34, U.S. Pat. Nos. 9,180,182 and 9,552,183. These sialic acid/siglec ligands and its polymers can be readily adopted for the current inventions.

In some embodiments, the formulation is applied to oral mucosa. It can be suppository, lozenge, tablet, film used for sublingual delivery and oral mucosa delivery. Current formulation used for sublingual delivery and oral mucosa delivery of allergen (e.g. pollen extract, dust mite extract) can be used with additional immune activity enhancing agent added to the formulation. Viscosity enhancing agent and/or mucosa adhesive agent can also be incorporated as described above. It can also contain therapeutically effective amount (e.g. the dose currently used in clinic) of anti-allergy drug such as Antihistamines, Corticosteroids, Mast cell stabilizers, and Leukotriene inhibitor as described above. It can also be a non-biodegradable container (e.g. a tablet made with plastic or metal) with small holes that allow the enclosed allergen/drugs to be released to the oral mucosa once it is placed in the mouth instead of the orally dissolvable tablet/lozenge from. A means that can prevent the formulation from being swallowed can be incorporated to the delivery system, such as a string, a band, a stick or tooth retainer. This will allow the removal of the formulation from mouth easily when severe allergy reaction is shown. For example, it can be in a format of a lollipop with the lozenge containing both allergen and other drugs. In one embodiment, a formulation is a mucosa adhesive tablet containing 0.1 mg-10 mg peanut protein, 3 mg imiquimod, and 3 mg Cetirizine with a wood handle attached in a lollipop format.

Compounds (e.g. the mixture, conjugate, polymer and nano/micro particle disclosed in the current invention) described herein can be administered as a pharmaceutical or medicament formulated with a pharmaceutically acceptable carrier. Accordingly, the compounds may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation Compounds may be formulated to include other medically useful drugs or biological agents. The compounds also may be administered in conjunction with the administration of other drugs or biological agents useful for the disease or condition to which the invention compounds are directed. The compound can be formulated in pharmaceutically acceptable carrier. As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the desired tissue or a tissue adjacent to the desired tissue. Pharmaceutically acceptable carriers are known to one having ordinary skill in the art may be used, including water or saline. As is known in the art, the components as well as their relative amounts are determined by the intended use and method of delivery. The compositions provided in accordance with the present disclosure are formulated as a solution for delivery into a patient in need thereof, and are, in some embodiments, focused on injection delivery.

Diluent or carriers employed in the compositions can be selected so that they do not diminish the desired effects of the composition. Examples of suitable compositions include aqueous solutions, for example, a saline solution, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents. In certain embodiments, a polymer matrix or polymeric material is employed as a pharmaceutically acceptable carrier. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, compositions provided herein may be formulated as films, gels, foams, or and other dosage forms. Suitable ionic strength modifying agents include, for example, glycerin, propylene glycol, mannitol, glucose, dextrose, sorbitol, sodium chloride, potassium chloride, and other electrolytes. Suitable pH buffering agents for use in the compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8.

In some embodiments the said composition/formulation further include a viscosity enhancing agent to increase its viscosity before or after being applied or orally taken or injected, which acts as a sustained release formulation. In certain embodiments, the injection has a viscosity greater than 10,000 cps at room temperature. In certain embodiments, the injection has a viscosity greater than 100,000 cps at room temperature. In certain embodiments, the injection has a viscosity greater than 5,000,000 cps at room temperature. In certain embodiments, the injection has a viscosity of 11,000,000 cps at room temperature. Example of the viscosity enhancing agent can be found readily from known pharmaceutical acceptable excipient such as hyaluronic acid, starch and carbomer. In some embodiments, the viscosity enhancing agent is biodegradable. The injection formulation can also be a thermal phase changing formulation. Thermal phase changing formulation is a formulation that change its phase from liquid at low temperature or room temperature (25° C.) to semisolid/gel when temperature increases to body temperature (37° C.), which can use poloxamer as excipient. A thermal phase changing injectable formulation can be given as either subcutaneous injection or intramuscular injections or intradermal injections to induce antigen specific immune tolerance and treat corresponding auto immune diseases or allergy. It has low viscosity at low or room temperature but high viscosity at body temperature. The preparation of this kind of high viscosity formulation and thermal phase changing injectable formulation can be adopted from related publications readily by the skilled in the art and are described previously in the current invention.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described. Dosage levels typically fall in the range of about 0.001 up to 1000 mg/dose; with levels in the range of about 0.05 up to 5 mg/dose are generally applicable. A compound can be administered parenterally, such as intramuscularly, subcutaneously, or the like. Administration can also be orally, nasally, rectally, transdermally or inhalationally via an aerosol. The compound may be administered as a bolus, or slowly infused. A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful initial doses in humans. Levels of drug in plasma may be measured, for example, by HPLC. The exact formulation, route of administration and dosage can be chosen by the individual physician in view of the patient's condition. In general, pharmaceutically effective and acceptable amount of antigen and immune activity enhancing agent in pharmaceutically acceptable formulation can be used, as long as it can produce satisfactory therapeutical (e.g. immune tolerance for allergen) effect, which can be determined experimentally by screening and testing with well-known protocol. This method can be used to treat antigen specific allergy.

For transdermal/transmucosal delivery or implant type formulation or oral formulation or sustained release formulation, the initial amount of allergy causing antigen and the amount of immune activity enhancing agent can be between 0.1-100 mg. When injection or non-sustained release oral formulation is used, the initial amount of allergen can be between 50 ug-5 mg and the amount of immune activity enhancing agent can be between 0.1-50 mg.

The formulation/composition can contain increased dose of allergen in later stage similar to the dosing protocol used by current treatment protocol using allergen (oral or topical or injection). That is, the treatment involves a series of formulations, the first formulation contains lowest amount of allergen and it gradually increases over time in the later formulation while the amount of other drug (e.g. immune enhancing agent) can be unchanged. The allergen amount in the first formulation can be the highest amount of allergen that can be tolerated by patient without causing severe allergenic reaction. In one example, the patient began with a first single dose of oral mucosal tablet formulation containing 0.1 mg of powdered egg white and 5 mg of imiquimod, after the initial dose, subject received approximately doubling doses of egg white but same amount of imiquimod every 30 minutes until the highest tolerated single dose is determined (as shown in table 1). Based on the highest tolerated single dose, subject begins daily dosing with between the formulation containing 25 and 200 mg of powdered egg white and 5 mg imiquimod, 1 dose daily for 2 weeks. As long as subject is tolerating current doses, the egg white powder in the formulation containing 5 mg imiquimod are increased by 25 mg every 2 weeks until reaching 150 mg and then increased by 50 mg every 2 weeks until reaching 300 mg. Once subjects reached the daily dose of 300 mg, they are instructed to take this daily dose that does not contain imiquimod for 2 years.

TABLE I

| | Powdered egg white, | Powdered egg white, |
|---|---|---|
| Dose no. | single dose (mg) | cumulative dose (mg) |
| 1 | 0.1 | 0.1 |
| 2 | 0.2 | 0.3 |
| 3 | 0.4 | 0.7 |
| 4 | 0.8 | 1.5 |
| 5 | 1.5 | 3.0 |
| 6 | 3.0 | 6.0 |
| 7 | 6.0 | 12 |
| 8 | 12 | 24 |
| 9 | 25 | 49 |
| 10 | 50 | 99 |
| 11 | 100 | 199 |
| 12 | 200 | 399 |

In the current application, the “/” mark means “and” and/or “or” and/or their combination. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from text books such as the chemistry textbooks, scientific journal papers and other well-known reference sources.

1. An injectable formulation to treat allergy comprising an antigen causing said allergy, an immune activity enhancing agent and a viscosity enhancing agent.

2. The injectable formulation according to claim 1, wherein the antigen is selected from antigen causing peanut allergy, antigen causing pollen extract, dust mite extract and egg white protein.

3. The injectable formulation according to claim 1, wherein the immune activity enhancing agent is Toll-like receptor agonist selected from imiquimod, poly IC and resiquimod.

4. The injectable formulation according to claim 1, wherein the formulation is a thermal phase changing formulation.

5. The injectable formulation according to claim 1 further comprising an anti-allergy agent selected from antihistamines.

6. A method for treating allergy in a subject, comprising administering to the subject an injection containing antigen causing said allergy, an immune activity enhancing agent selected from poly IC, resiquimod, imiquimod, and a viscosity enhancing agent.

7. The method according to claim 6, wherein the antigen is selected from antigen causing peanut allergy, antigen causing pollen extract, dust mite extract, pollen extract and egg white protein.

8. The method according to claim 6, wherein the injection further comprising an antihistamine.

9. The method according to claim 6, wherein the injection is a thermal phase changing formulation.
